(19) United States
(12) Patent Application Publication
(10) Pub. No.: US 2024/0013472 A1
(43) Pub. Date: Jan. 11, 2024

(54) Surface Determination Systems, Threat Detection Systems and Medical Treatment Systems

(71) Applicant: Battelle Memorial Institute, Richland, WA (US)

(72) Inventors: Richard Trevor Clark, West Richland, WA (US); David M. Sheen, Richland, WA (US)

(73) Assignee: Battelle Memorial Institute, Richland, WA (US)

(21) Appl. No.: 18/019,631

(22) PCT Filed: Apr. 8, 2021
(86) PCT No.: PCT/US2021/026471

§ 371 Date: Feb. 3, 2023

(51) Int. Cl. G06T 15/08; G06T 7/73; G01S 13/90; A61N 1/40

(52) U.S. Cl. CPC G06T 15/08; G06T 7/73; G06T 7/74; G01S 13/9064; A61N 1/403; G06T 2207/10044; G06T 2207/10028; G06T 2207/30088; G01S 13/887

(57) ABSTRACT

Surface determination systems, threat detection systems and medical treatment systems are described. According to one aspect, a surface determination system includes processing circuitry configured to access a three-dimensional complex-valued image volume of a target, define image locations of the image volume using first and second dimensions of the image volume, for each of the first locations, identify voxels along a third dimension of the image volume that correspond to the respective first location, for each of the first locations, select one voxel that corresponds to the first location as having an increased amplitude compared with other voxels that correspond to the first location, and for each selected voxel, use phase information to identify a second location in the third dimension for the selected voxel that corresponds to a location of a surface of the target and that is different than a third location of the respective voxel.

### STATEMENT AS TO RIGHTS TO INVENTIONS MADE UNDER FEDERALLY-SPONSORED RESEARCH AND DEVELOPMENT

This invention was made with Government support under Contract DE-AC05-76RL01830 awarded by the U.S. Department of Energy. The Government has certain rights in the invention.

### TECHNICAL FIELD

This disclosure relates to surface determination systems, threat detection systems and medical treatment systems.

### BACKGROUND OF THE DISCLOSURE

Active microwave and millimeter-wave (mm-wave) radar imaging has been deployed for a variety of applications including personnel screening, in-wall imaging, through wall imaging, and ground penetrating radar in but a few illustrative examples. Optically opaque 20 low loss dielectrics are nearly transparent to microwaves and mm-waves which makes them ideally suited for various applications to scan through these low loss dielectrics and generate images of contents therein. As a result, radar imaging has become ubiquitous for airport screening using methods such as cylindrical mm-wave imaging techniques or multistatic array techniques.

At least some aspects of the present disclosure are directed towards apparatus and methods for determining a surface of a target from radar images. Additional aspects are of the disclosure are disclosed below including example embodiments of a threat detection systems and medical treatment systems.

### BRIEF DESCRIPTION OF THE DRAWINGS

Example embodiments of the disclosure are described below with reference to the following accompanying drawings.

FIG. 1 is a functional block diagram of a surface determination system according to one embodiment.

FIG. 2 is an illustrative antenna array of a surface determination system according to one embodiment.

FIG. 3 is an illustrative representation of scanning operations with respect to a target according to one embodiment.

FIG. 4 is a three-dimensional radar magnitude image in the form of a rectangular cuboid with principal projections on each face of the rectangular cuboid.

FIG. 5 is a flow chart of an example method of generating a representation of a surface of a target from an image volume according to one embodiment.

FIG. 6 is an illustrative representation of a plurality of projections through a three-dimensional complex-valued image volume according to one embodiment.

FIG. 7 is an illustrative representation of an antenna system of a threat detection system according to one embodiment.

FIG. 8 is an illustrative representation of a medical treatment system according to one embodiment.

### DETAILED DESCRIPTION OF THE DISCLOSURE

Some aspects of the present disclosure improve upon the state of the art by carefully focusing radar images to preserve phase information inherent in the propagation of the electromagnetic waves used to form the radar images. In some implementations, wideband microwave or millimeter-wave electromagnetic waves are used for scanning and generating radar images. Thereafter, phase information of reconstructed radar images may be used to determine locations of a surface of a target since phase follows the surface of the target. In particular, surfaces of constant phase, such as zero-phase, in the reconstruction follow the contours of the body or target. Furthermore, the surface of the target tracks the zero-phase contour precisely if the image reconstruction is performed in an exacting manner as described herein. Accordingly, surfaces of a target can be estimated by forming a high-resolution image using backprojection or similar methods and then finding the surface by numerically finding the zero-phase position over a lattice of positions.

High-resolution active wideband microwave and millimeter-wave imaging systems may be formed by mechanically, or electronically scanning a transceiver over a 2D aperture. A transmitting portion of a transceiver emits a wideband signal that interacts with the target and is captured coherently by a receiver portion of the transceiver in one embodiment at each point in the aperture. The subsequent data is three-dimensional (3D) consisting of two spatial axes and one frequency axis in the described embodiment. This data can then be focused using backprojection or other similar methods. Resolution in microwave imaging is limited by diffraction in the lateral dimensions and by bandwidth in the range or depth dimension.

Conventional techniques for tracking the surface are typically done after image formation by taking the magnitude image and forming iso-surfaces, or surfaces of constant amplitude. However, this process causes errors in the surface estimation since it inherently assumes that brightness is related to position and a brighter zone in the image will appear closer than a dimmer zone, even if they are at the same depth. Brightness also depends on the orientation of the image target relative to the image aperture.

Aspects of the disclosure discussed herein achieve high accuracy by eliminating bias caused by image amplitude variations and by exploiting the image phase. The image phase varies approximately 360 degrees for every half-wavelength in depth variation and the zero-phase position can be estimated to accuracies of better than a few degrees according to some embodiments disclosed herein. Therefore, the surface of a target can be estimated to a small fraction of one-half wavelength using inventive embodiments described herein while conventional methods are limited by the depth resolution, which is typically much larger than one-half wavelength.

The image reconstruction of some of the disclosed embodiments preserves the phase and samples the image volume finely around the target to generate a three-dimensional image volume about the target. At least some of the inventive embodiments project along a line through the image volume in a specified direction and estimate the zero-phase position with the highest complex amplitude or magnitude along each projection or line corresponding to the specified direction. The location of this point closely approximates the position of the surface of the target along each line or projection. In some embodiments, the image volumes are used to generate representations of a surface of the target that was scanned. In more specific embodiments, the image volumes are each reduced to a collection of three-dimensional points, such as a point cloud, that closely approximates the surface of the target.

In some embodiments discussed below, the surface of the target, or a portion of the surface, can be tracked over time through an optimization process that estimates a coordinate transformation required to optimally align two point clouds corresponding to locations of the surface of the object at different moments in time. A point-to-plane iterative closest point (ICP) algorithm may be used to estimate the coordinate transformation in some implementations described below. However, once the point clouds are generated there are many different options to calculate the alignment between point cloud surfaces. For example, a surface mesh may be generated from a surface point cloud and then used to register two surfaces in one other illustrative example.

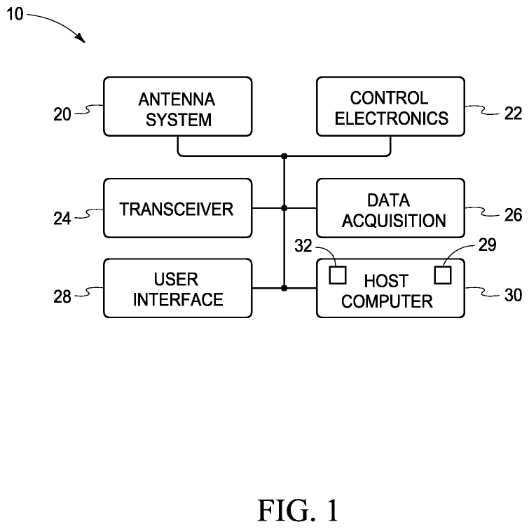

Referring to FIG. 1, components of an example embodiment of a surface determination system 10 are shown. The illustrated system 10 includes an antenna system 20, control electronics 22, a transceiver 24, a data acquisition system 26, a user interface 28, and a host computer 30. Additional arrangements of system 10 are possible including more, less and/or alternative components.

Antenna system 20 comprises a plurality of transmitters which are configured to emit electromagnetic energy towards a target being scanned. The transmitters of antenna system 20 emit the electromagnetic energy responsive to electrical signals received from transceiver 24. Antenna system 20 further comprises a plurality of receivers which are configured to receive electromagnetic energy reflected from the target and to output electrical signals to the transceiver 24 that correspond to the received electromagnetic energy.

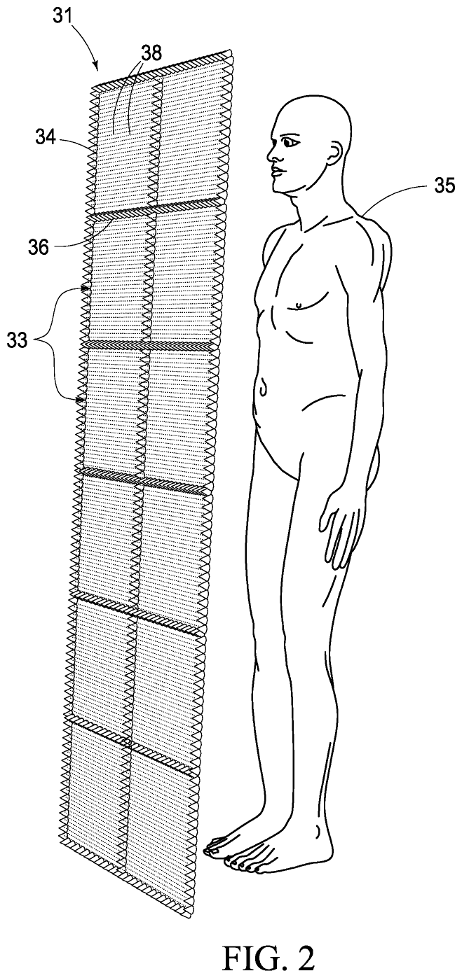

Antenna system 20 may additionally include a switching network or matrix to selectively choose different pairs of transmit and receivers to define a plurality of sample points in space in some embodiments. In other embodiments, the transmitters and receivers may be moved during scanning operations including the transmitting and receiving of electromagnetic signals. Details regarding an example configuration of an antenna array of the antenna system 20 that may be used are shown in FIG. 2.

Control electronics 22 are configured to control transmit and receive operations of antenna system 20, including switching of antennas of the transmitters and receivers therein, as well as operations of transceiver 24 and data acquisition system 26.

Transceiver 24 is coupled with the antenna system 20 and configured to apply electrical signals to the antenna system 20 to generate the transmitted electromagnetic waves and to receive electrical signals from the antenna system 20 corresponding to received electromagnetic waves. Transceiver 24 is coherent where the local carrier of the receiver thereof is phase locked with the carrier of the transmitter of the transceiver 24.

The data acquisition system 26 acquires and digitizes the transceiver output data. The data acquisition system 26 also buffers the transceiver output data and sends it to the host computer 30.

User interface 28 includes a computer monitor configured to depict visual images for observation by an operator, for example, including images generated from the radar scanning and revealing concealed contents upon an individual. User interface 28 is additionally configured to receive and process inputs from the operator. In some embodiments, host computer 30 uses automated threat detection algorithms to inspect the generated imagery for threats.

Host computer 30 includes processing circuitry 29 configured to perform or control various operations of system 10. In one embodiment, processing circuitry 29 is arranged to process data, control data access and storage, issue commands, and control other desired operations. Processing circuitry 29 may comprise circuitry configured to implement desired programming provided by appropriate computer-readable storage media in at least one embodiment. For example, the processing circuitry 29 may be implemented as one or more processor(s) and/or other structure configured to execute executable instructions including, for example, software and/or firmware instructions. Other exemplary embodiments of processing circuitry 29 include hardware logic, GPU, PGA, FPGA, ASIC, state machines, and/or other structures alone or in combination with one or more processor(s). These examples of processing circuitry 29 are for illustration and other configurations are possible.

In one embodiment, processing circuitry 29 performs waveform signal processing and calibration and processes received radar data to generate radar images of the target. The host computer 30 may be implemented as a high-performance PC workstation that supports fast image reconstruction and processing that exploits parallel processor architecture of modern computers in one more specific embodiment.

Host computer 30 also includes storage circuitry 32 configured to store programming such as executable code or instructions (e.g., software and/or firmware) used by the host computer, electronic data, databases, radar data, image data, or other digital information and may include computer-readable storage media. At least some embodiments or aspects described herein may be implemented using programming stored within one or more computer-readable storage medium of storage circuitry 32 and configured to control appropriate processing circuitry 29 of the host computer 30.

The computer-readable storage medium may be embodied in one or more articles of manufacture which can contain, store, or maintain programming, data and/or digital information for use by or in connection with an instruction execution system including processing circuitry 29 in the exemplary embodiment. For example, exemplary computer-readable storage media may be non-transitory and include any one of physical media such as electronic, magnetic, optical, electromagnetic, infrared or semiconductor media.

Referring to FIG. 2, an example antenna array 31 of the antenna system 20 is shown according to one embodiment. The illustrated antenna array 31 is a sparse array that includes a plurality of square unit cells 33 with plural transmitters 34 along the vertical edges and plural receivers 36 along the horizontal edges arranged in a grid. In another embodiment, the transmitters are arranged horizontally and the receivers are arranged vertically in a grid. Within a given unit cell 33, all combinations of transmitters 34 and receivers 36 are selected in pairs and used to effectively raster scan across the aperture where an effective sample location 38 is the midpoint between the transmitter 34 and receiver 36 of a selected pair.

For a selected pair of transmitters 34 and receivers 36, the transceiver is used to produce a swept wideband microwave or millimeter-wave signal that is radiated by the transmitter 34 of the selected pair. This signal interacts with the imaging target 35, such as a human body in the illustrated example, and is reflected and received by the transceiver through the receiver 36 of the selected pair.

In one embodiment, surface determination system 10 implements three-dimensional radar imaging by transmitting and receiving a swept frequency signal over a sampled two-dimensional aperture, such as the planar aperture shown in FIG. 2. The aperture may have other shapes, such as cylindrical, in other embodiments.

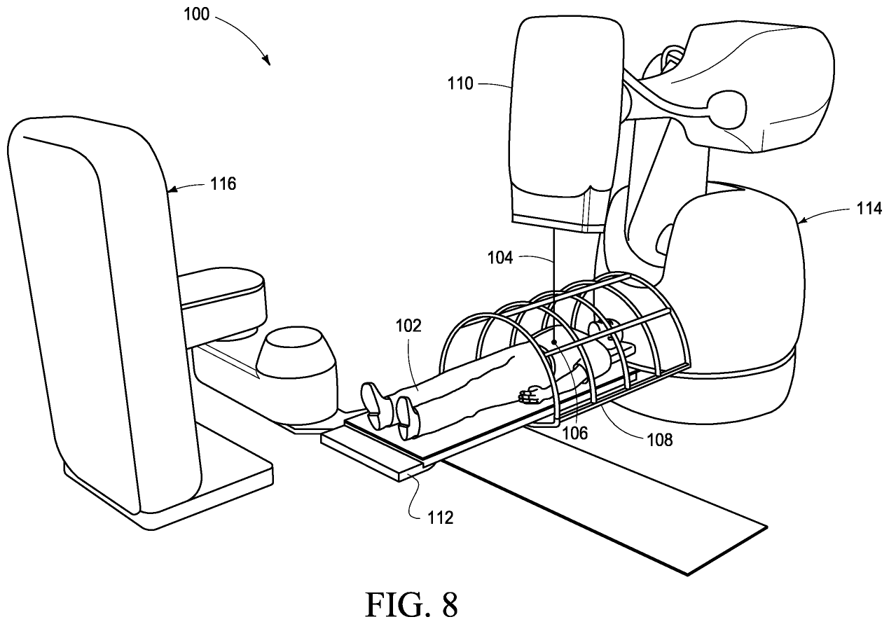

Generated raw radar data from the scanning is fully three-dimensional with two effective aperture or spatial axes and one frequency axis. An image reconstruction algorithm (such as backprojection) can then be used to focus the radar data to generate a 3D image of the target 35. The sparse nature of the radar array could allow for radiation to be delivered to a patient through the voids in the unit cells 33, for example, as discussed below with respect to the medical treatment system of FIG. 8.

The depth resolution is inversely proportional to the swept frequency bandwidth and the lateral resolution is obtained by scanning over the 2D aperture. In one embodiment, the swept frequency bandwidth of a continuous wave signal is 1-100 GHz although other microwave or millimeter ranges may be used, such as 10-40 GHz. The processing circuitry processes the raw image data to mathematically focus the radar data into a three-dimensional complex-valued image of the target's reflectivity. This is commonly done with methods that use a Fast Fourier Transform (FFT) due to its extremely high numerical efficiency as discussed in D. Sheen, D. McMakin, and T. Hall, “Near-field three-dimensional radar imaging techniques and applications,” Appl. Opt., AO, vol. 49, no. 19, pp. E83-E93, July 2010, the teachings of which are incorporated herein by reference.

As mentioned above, backprojection may be used to mathematically focus radar data. Backprojection is similar to a multi-dimensional correlation and may be implemented using a graphical processing unit (GPU) in one example. Additional details regarding backprojection are discussed in D. L. Mensa, High Resolution Radar Cross-section Imaging, Artech House, 1991, the teachings of which are incorporated herein by reference. In addition, the formation of a three-dimensional complex-valued image volume from raw radar data using backprojection according to an example embodiment is discussed below.

In this described embodiment, a generalized synthetic aperture focusing technique for microwave and millimeter-wave imaging, also referred to as range-domain backprojection, can be formulated as:

$$v ( x , y , z ) = {\sum }_{{a}_{1}} {\sum }_{{a}_{2}} w ( {a}_{1} , {a}_{2} ) s ( {a}_{1} , {a}_{2} , r ) {e}^{j 2 {k}_{c} r} \qquad Eqn . ( 1 )$$

where v is the complex image amplitude at location (x,y,z), s(a₁,a₂,r) is the radar range-domain phase-history from aperture location (a₁, a₂) at range r, k_{c} is the wavenumber at the center frequency, and w(a₁, a₂) is a weighting function applied over the two dimensions of the aperture to reduce side lobe levels. The range-domain radar phase history, s(a₁,a₂,r), is obtained by taking the inverse Fourier transform of the radar phase history, S(a₁,a₂,ƒ), and multiplying by a correction factor e^{j2k}^{₁}^{r}e^{-j2k}^{ᵣ} to correct the phase of the range-domain waveforms and reduce fast phase variation to allow for accurate interpolation as shown in Eqn. 2:

s(a₁,a₂,r)={IFFT(w(ƒ)S(a₁,a₂,ƒ))e^{j2k}^{₁}^{r}^{ₙ}e^{-j2k}^{_{c}}^{r}^{ₙ}}|ᵣ Eqn. (2)

where the wavenumber at the start frequency is k₁ and frequency window function w(ƒ) is used to control sidelobes in range. One example window function that may be utilized is a Hamming window.

The range-domain back projection algorithm essentially multiplies the response from each aperture location, s(a₁,a₂,r), with the complex conjugate of the expected response from a scatterer at a voxel at location (x,y,z) and range r, e^{j2k}^{_{c}}^{r}. If there is truly a scatterer at that voxel location, the actual response will be multiplied by its conjugate resulting in a zero-phase or real value which when summed across the entire aperture will all add in phase creating a large magnitude at a point of zero-phase. Locations where there is not a scatterer will add values with fluctuating phase that will decorrelate and the magnitude will tend to zero.

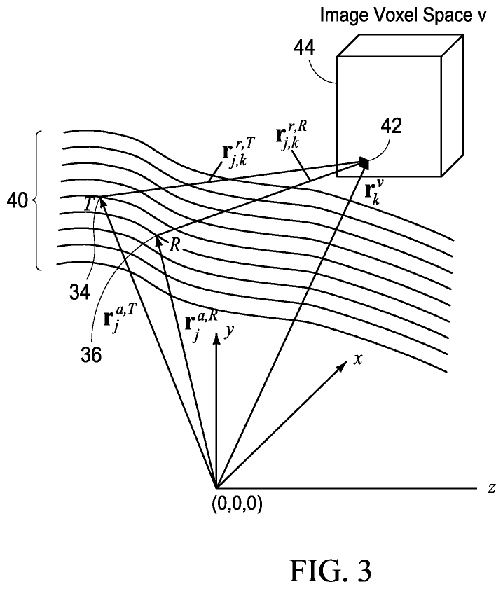

Referring to FIG. 3, an illustrative representation of scanning a target (not shown) and use of a range-domain back projection algorithm is shown. Radar transmitters 34 and receivers 36 are scanned either electronically as discussed above, or alternatively mechanically, over a typically planar or cylindrical aperture 40, to implement scanning of an image voxel space 44 about the target to be scanned. The 3D radar phase history, two spatial axes and a frequency axis, can be used to focus and generate a radar image in the form of a 3D complex-valued image volume. The image volume includes a plurality of voxels 42 each having an associated complex value that includes an amplitude and phase. In FIG. 3, a selected pair including transmitter 34 and a receiver 36 located at positions T, R emit and receive electromagnetic energy with respect to an illustrative voxel 42 and a plurality of ranges between the transmitter 34 and receiver 36 and voxel 42 are shown as well as the ranges of the transmitter 34 and receiver 36 with respect to the origin.

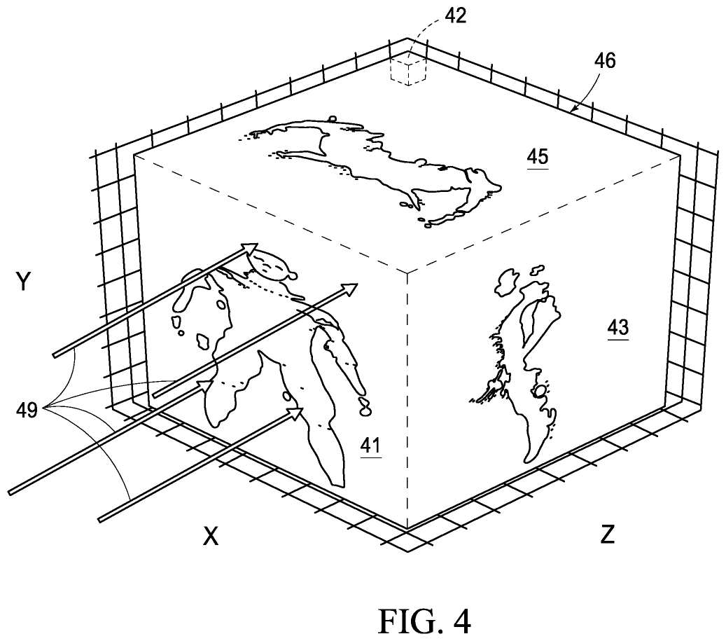

The above-described range-domain backprojection is used in one embodiment to focus the radar-phase history data into a 3D complex-valued image volume, an example of which is shown in FIG. 4 as a result of scanning a human target.

The depicted image volume 46 is in the form of a rectangular cuboid that corresponds to the image voxel space 44 in the illustrated embodiment and includes a plurality of complex-valued voxels 42 defined by the X, Y, Z axes or dimensions. FIG. 4 depicts a radar magnitude image of a human target with the principal projections on faces 41, 43, 45 corresponding to the front, top and right side of the rectangular cuboid, respectively. The voxel 42 shown in FIG. 4 is illustrative and larger than actual voxels of the image volume (i.e., a generated image volume includes many more voxels than the illustrative example shown in FIG. 4).

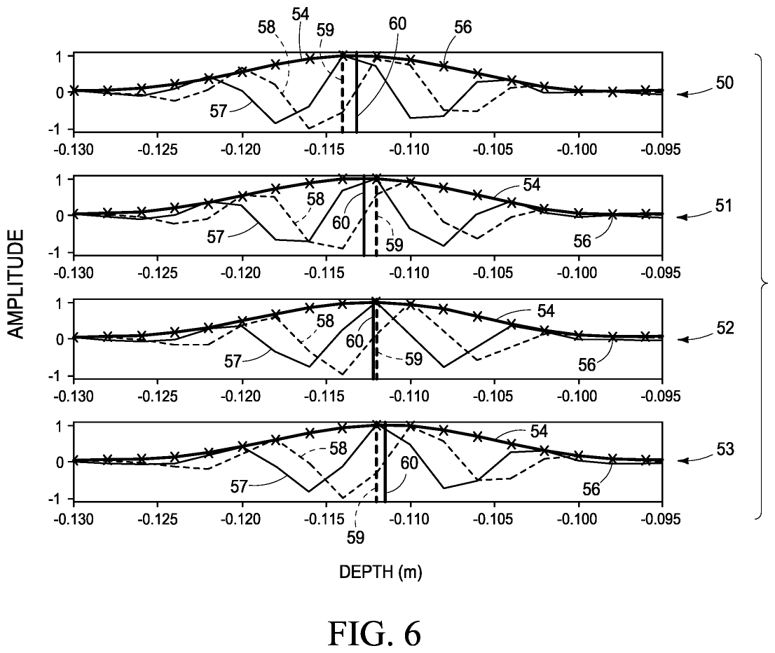

For each X and Y image location in surface 48, the processing circuitry projects 49 through the Z (e.g., depth) direction to find the voxels having increased complex amplitude values along the projection as discussed further below with respect to FIG. 6. Each projection 49 is a straight line perpendicular to face 41 of the image volume 46. A given projection 49 through an image volume identifies all Z values of the image volume in the depth direction that correspond to a given X-Y image location. As discussed further below, one of the voxel values in the depth direction of a projection 49 is interpolated to identify a point of a surface of a target being scanned that corresponds to the given X-Y location.

As discussed above, the actual response at a given voxel location will be multiplied by its complex conjugate resulting in a real value which when summed across the entire aperture will all add in phase creating a large magnitude at a point of zero-phase in the presence of a scatterer at the given voxel location and locations where there is not a scatterer will add values with fluctuating phase that will decorrelate and the magnitude will tend to zero. This implies that a surface of a target will be at a location near the maximum image amplitude at the zero-phase location of the complex voxel amplitude. By projecting through the complex-valued image volume and finding the zero-phase location under the maximum complex amplitude envelope along the projection 49, a point cloud or other representation of the target surface can be generated that is largely independent of image amplitude variations.

In some embodiments discussed below, the amplitude of the complex-valued image only affects which points are valid surface points based on a chosen amplitude or magnitude threshold. For the case where the Z direction is depth, a point for each X, Y image location in the complex volume 46 may be used to generate a point cloud for the image volume if the point has an amplitude above the threshold as discussed further below.

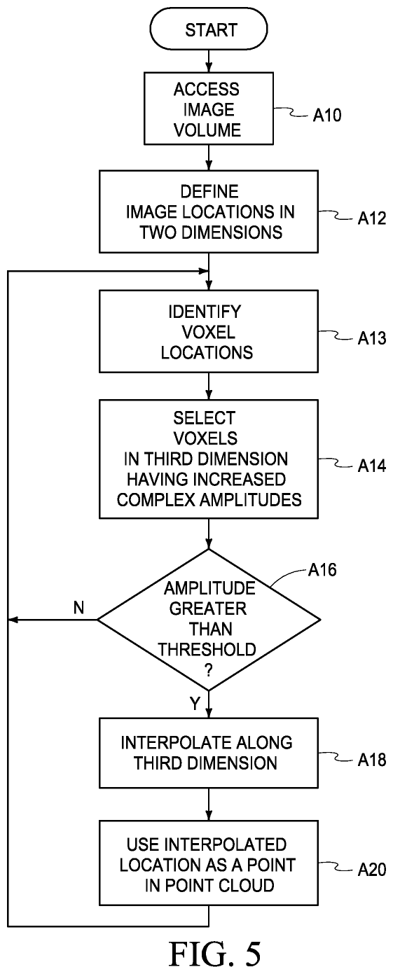

Referring to FIG. 5, a flow chart of an example method of processing one or more radar images of a target to determine a plurality of points, for example of one or more point clouds, that correspond to locations of a surface of the target in space when the one or more radar images where generated. As discussed below, amplitude and phase information of complex values of the radar image in the form of a three-dimensional complex-valued image volume are used to generate a representation of the surface of the target, such as a point cloud. The illustrated method may be executed using processing circuitry of the host computer described above in one embodiment. Other methods are possible including more, less and alternative acts.

At an act A10, data of a previously generated three-dimensional complex image volume is accessed. The image volume may have been generated using backprojection and be in the shape of a rectangular cuboid according to the example embodiment discussed above. The accessed data of the image volume includes complex values of amplitude information and phase information for each of the voxels within volume.

At an act A12, a plurality of image locations of the image volume are defined. Two spatial dimensions or axes (e.g., X and Y) of the accessed image volume are utilized to define the image locations in the described example.

At an act A13, a plurality of voxels are identified along a third dimension (e.g., Z) for each of the X, Y image locations. A straight line projection that is perpendicular to the X, Y face of the rectangular cuboid is made through the image volume in the Z (depth) dimension of the image volume for each of the defined X, Y image locations to identify a plurality of voxel locations in the Z dimension of the image volume that correspond to the respective X, Y image location. For a given X, Y location, a complex amplitude value and phase value for each voxel location corresponding to the given X, Y location in the depth direction of the image volume is retrieved.

At an A14, the retrieved voxels of the projection in the depth direction are processed to identify voxels in each projection which have increased complex amplitudes compared with other voxels of the respective projection and the selected voxels may be used to define a maximum complex amplitude envelope for the given projection. The voxel for each projection having an increased complex amplitude compared with other voxels of the same projection is selected as a result of the processing in act A14. In a more specific embodiment, a voxel having the maximum complex amplitude is selected for each projection.

At an act A16, the complex amplitude of the voxel of a projection for a given X, Y image location having the maximum complex amplitude and selected using act A14 is compared with a threshold.

The voxels of the projection are disregarded and not utilized with respect to surface determination of the target if the selected voxel having the maximum complex amplitude does not exceed the threshold (and is therefore deemed to not correspond to the surface of the target). Thereafter, the method returns to act A13 to process voxel values of another projection through the image.

The method proceeds to an act A18 if the complex amplitude of the voxel processed in act A16 exceeds the threshold. The voxel values under the maximum complex amplitude envelope are interpolated at act A18 using phase information of the voxel values to identify an interpolated value that corresponds to the surface of the target. For example, as discussed below with respect to FIG. 6, the interpolated value may correspond a location in the Z dimension direction that has a given phase value, such as zero-phase, and is closest to a voxel location having a maximum complex amplitude for the projection. The use of interpolation increases the resolution of the surface determination of the target in the third dimension compared with use of the voxel having the maximum complex amplitude without interpolation since the interpolated value having to the given phase value and identified as corresponding to the surface of the target is often between the locations of two adjacent voxels in the projection. Accordingly, the interpolated locations corresponding to the given phase value more accurately correspond to the actual locations of the surface of the target compared with locations of the voxels having the increased complex amplitude.

At an act A20, the location (i.e., depth) resulting from the interpolation for the given projection is utilized to generate a representation, such as a point cloud, of the surface of the target. Thereafter, the method returns to act A13 to process voxel values of another projection. Using the above-described example process, only voxels having complex amplitudes greater than the threshold are used to generate the representation of the surface of the target.

Referring to FIG. 6, four successive projections 50-53 through the complex image volume moving horizontally are graphically shown for four different respective X-Y image locations of an image volume. Each value depicted has been normalized to unit amplitude.

Line 54 in each projection corresponds to the complex magnitude or amplitude of the image volume at each voxel 56 (sample point) for the respective projection. Line 57 in each projection is the real part of the complex image for the respective projection, and line 58 in each projection is the imaginary part of the complex image for the respective projection.

The vertical line 59 of each projection is the voxel location of the maximum complex amplitude along the respective projection.

The vertical line 60 of each projection is a location that results from interpolation using phase information of the image volume. In one embodiment, phase information of the voxels is used to identify an interpolated location in the third dimension for each of the X-Y locations that corresponds to a surface of the target and that is different than the locations of the voxels. In one embodiment, a given phase value of zero-phase is used to identify the interpolated locations in the third dimension for each of the X-Y image locations. In one embodiment, the interpolated location in the third dimension for a given X-Y image location is a zero-phase location closest to the voxel having the maximum complex amplitude for the given X-Y location. In particular, line 60 for each projection is the zero-phase location that is closest or nearest to the maximum complex amplitude of line 59 and is selected as a location or point corresponding to a surface of the target being imaged for the depth direction for that respective X-Y location and projection. Accordingly, the interpolated location for the given X-Y location is selected to be the zero-phase position closest to the maximum complex amplitude. As mentioned above, X-Y locations that do not have a complex amplitude above the given threshold are identified as not corresponding to the surface of the target. In addition, it is also possible that the zero-phase location of a given projection may also correspond exactly to the maximum amplitude location of the projection and be used to generate a representation of a surface of a target.

In some embodiments, the interpolated locations (i.e., depths) for the X-Y image locations may be used by the processing circuitry to generate a representation of the surface of the target. For example, the representation of the surface of the target may be a point cloud although other embodiments are possible.

In some arrangements, the phase value of interest utilized during the interpolation may be a value other than zero and utilized to identify the locations of the surface of the target for the different X-Y image locations. For example, other or different image reconstruction techniques and/or different processing of the radar data may be utilized to generate an image volume in other embodiments and may result in a different constant phase value (apart from zero) that corresponds to a surface of the target and may be used during the interpolation operations described above to locate points for inclusion in the point cloud or other representation of the surface of the target being scanned.

Processing of the original complex-valued three-dimensional radar image enables the generation of a smooth and accurate point cloud representation of the surface of an imaged target by proper exploitation of the phase information as discussed above. Use of phase information of the image allows decoupling of the magnitude of the image from the geometry of the target thereby allowing the surface of the target to be determined with increased accuracy compared with arrangements that solely rely upon use of magnitude information to determine the surface of the target.

In particular, as shown in the projections of FIG. 6, the determined zero-phase locations vary in a smooth predictable way as the projection moves along different lines in the 3D volume compared with maximum amplitude locations that are more erratic.

Pseudocode of an example zero-phase surface estimation algorithm that is configured to select the zero-phase crossing near the maximum amplitude as the location of the surface of a target for inclusion as a point in a point cloud for a respective X-Y location is shown below:

```
for i in range(nx):
  for j in range(ny):
    ${z}_{max} = {arg max}_{z} ( abs ( v [ i , j , z ] ) )$
    maxValue = abs(v[i, j, zₘₐₓ])
    if maxValue ≥ threshold:
      z′ = interpolate the complex amplitude, v[i, j], around zₘₐₓ
        to find the closest point where angle(v[i, j, z′]) = 0
      PointCloud[i, j] = z′
```

As discussed above, locations of zero-phase in the depth direction of a generated 3D image volume may be utilized to locate a surface of a target since the zero-phase information is largely independent of image amplitude variations. Ideally a surface estimation of a target should be independent of the object's orientation, however, the amplitude response of an object in a microwave or millimeter-wave radar image is dependent not only on the target's geometry, but also on its orientation relative to the radar array. An advantage of using a point cloud based on the zero-phase location compared with use of amplitude information only of 3D images is that the geometry of the objects in the image is decoupled from the image amplitude.

A wide variety of new applications and processing techniques are enabled once a representation, such as a point cloud, has been generated from the surface of a target. For example, point clouds may be generated for use in threat detection, such as monitoring for weapons or contraband in screening of persons at a public venue, such as an airport, stadium event, etc. A point cloud derived surface of a person shows more information than an intensity projection image and includes information about the geometry of the target image that does not depend on the image intensity or orientation of the target relative to the antenna array. This provides more information for anomaly detection, such as contraband or weapons concealed beneath clothing of an individual.

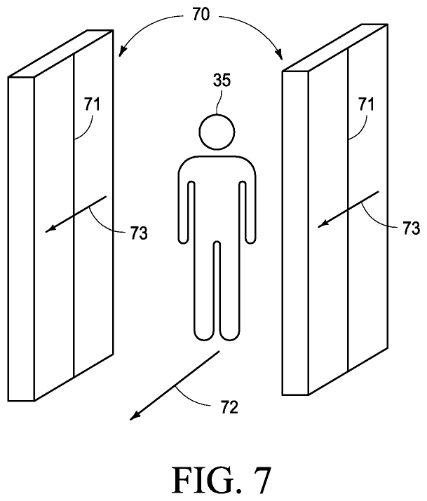

Referring to FIG. 7, an antenna system of a threat detection system including a plurality of antenna array columns 70 are shown in a 2D scanner configuration according to one embodiment. The example threat detection system may be implemented in a walk-by imaging application, for example to scan for concealed threats or contraband upon clothed individuals entering a screened area. The columns 70 are arranged opposite to one another and positioned to scan opposite sides of a target 35 moving on a path 72 between columns 70. The columns are configured to emit electromagnetic energy towards target 35 moving on path and receive electromagnetic energy reflected from the individual. Electromagnetic energy of millimeter wave or microwave frequencies may be utilized for the scanning and which enable scanning of the individual to reveal threats or contraband concealed by the individual's clothing.

Each column 70 includes a linear antenna array 71 that includes both transmit and receive antennas (not shown in FIG. 7) in one embodiment. The linear antenna array 71 in each column 70 is mechanically moved 73 next to the target 35 during scanning of the target 35. A length of the linear antenna array 71 is one spatial dimension of the aperture and movement 73 of the linear array 71 is a second spatial dimension of the aperture. Real-time, high-speed data collection and scanning is used in one embodiment to effectively freeze the motion of the target 35 during a data frame from each column 70 and to allow fine sampling of the target 70 passing through the system.

In another embodiment, the columns 70 each include a 2D antenna array such as shown in FIG. 2 that electronically scans a two-dimensional aperture to freeze motion.

Numerous transmit locations may be provided along the length of the column 70 for angularly diverse illumination of the target 35. In one embodiment, the sequentially switched linear array scans one dimension of the imaging aperture electronically at high speed and is accomplished by sequencing through each transmit and receive pair of antennas using microwave- or millimeter-wave switching networks connected to the radar transceiver. Data is continuously collected as the target 35 moves adjacent to or through the scanning system.

In one embodiment, a sparse array technique is utilized which achieves required sampling density with a reasonable number of antennas by using multiple combinations of transmit and receive antennas to increase the density of aperture samples while reducing the number of antenna elements. Details regarding suitable antenna arrays including sparse arrays are described in U.S. Pat. No. 8,937,570 and Sheen, DM, “Sparse Multi-Static Arrays for Near-Field Millimeter-Wave Imaging,” In 2013 IEEE Global Conference on Signal and Information Processing, GlobalSIP, IEEE Computer Society, pp. 699-702, 2013, the teachings of which are incorporated herein by reference.

The threat detection system may include additional components such as shown in FIG. 1 to implement scanning operations of an individual as well as processing of radar data to generate image volumes and processing of the image volumes to determine points of a surface of the target 35. In one embodiment, the processing circuitry uses the received electromagnetic energy to generate a three-dimensional complex-valued image volume of at least part of the clothed individual. The processing circuitry is further configured to process amplitude information and phase information of the complex values to generate a representation, such as a point cloud, of a surface of the target 35 to provide information regarding a surface anomaly beneath clothing of the clothed individual. In one embodiment, the processing circuitry may control the user interface to display a graphical image of the point cloud corresponding to the surface of the target 44.

Based on an accurate surface representation of an imaged object or person it is possible to look at how the surface changes spatially using gradients. Unnatural or sharp changes might indicate a threat that could be detect. For example, a manmade object should have easily identifiable characteristics that are distinct from the natural shape of the body.

In addition, it is possible to register point-clouds between radar images generated from scans of a target at different moments in time to provide information regarding movement of the surface of the target between the moments in time when the radar images were captured. An accurate surface allows matching of objects based on their geometry independent of the image amplitude.

Different methods may be used to register two different point clouds, for example, including use of an Iterative Closest Point algorithm (ICP), or generating a surface mesh and aligning surfaces as discussed in S. Rusinkiewicz and M. Levoy, “Efficient variants of the ICP algorithm,” in Proceedings Third International Conference on 3-D Digital Imaging and Modeling, May 2001, pp. 145-152, and M. A. Audette, F. P. Ferrie, and T. M. Peters, “An algorithmic overview of surface registration techniques for medical imaging,” Medical Image Analysis, vol. 4, no. 3, pp. 201-217, September 2000, the teachings of which are incorporated herein by reference. In another embodiment, a variant of the ICP algorithm referred to as point-to-plane ICP algorithm from the Open3D python library may be used as discussed in Q. Y. Zhou, J. Park, and V. Koltun, “Open3D: A Modern Library for 3D Data Processing,” arXiv, 2018, the teachings of which are incorporated herein by reference.

The general ICP algorithm iteratively minimizes an objective function, ƒ, by updating a transformation matrix, T, to align two point clouds as discussed in P. J. Besl and N. D. McKay, “A method for registration of 3-D shapes,” presented at the IEEE Transactions on Pattern Analysis and Machine Intelligence, February 1992, and Y. Chen and G. Medioni, “Object modeling by registration of multiple range images,” in Proceedings of the IEEE International conference on Robotics and Automation (ICRA), (Sacramento, CA, USA), pp. 2724-2729, April 1991, the teachings of which are incorporated herein by reference. This objective function is the minimization of the distance between points in a correspondence set, (p,q)∈K, between a source point cloud, q∈Q, and a target point cloud, p∈P. The point-to-plane ICP variation's objective function utilizes an estimated surface normal, nₚ, to penalize corresponding points that are tangential to the estimated surface as discussed in the Chen reference incorporated by reference above. The objective function to be minimized is formulated as shown in Equation 3:

$$f ( T ) = {\sum }_{( p , q ) ∈ K} {( ( p - Tq ) \cdot {n}_{p} )}^{2} \qquad Eqn . ( 3 )$$

This method does not assume there is a 1:1 correspondence between all points in the two-point clouds. It only minimizes the error between points that are determined to have correspondence that are useful in some embodiments because based on the orientation of an object when it is imaged there could be shadowing of the surface creating “holes” in the point cloud that may not be there when the object is in a different orientation. The point-to-plane ICP algorithm was found to provide millimeter and sub-millimeter level registration accuracy during simulated and experimental test cases.

In some embodiments, a rigid transformation between two-point clouds is assumed, although non-rigid registration methods that do not make this assumption may be used as discussed in L. Liang et al., “Nonrigid iterative closest points for registration of 3D biomedical surfaces,” Optics and Lasers in Engineering, vol. 100, pp. 141-154, January 2018, the teachings of which are incorporated herein by reference.

The algorithm outputs a transformation matrix that is indicative of movement of the surface of the target between the different radar images in six degrees of freedom including three corresponding to rotational movement and three corresponding to translation movement. The determined movement or motion of the surface may be used in different applications including monitoring movement of a target surface (i.e., skin of a patient) for use in medical implementations in one illustrative example.

Referring to FIG. 8, a medical treatment system 100 is shown according to one embodiment. The illustrated system 100 is configured to deliver a therapeutic treatment 104, such as radiation or ultrasound pulses, to a patient 102 undergoing medical treatment. The determination of surfaces as described above may be used to control the delivery of the therapeutic treatment 104 to a specific desired target location 106 of the skin of the patient 102 during the delivery of therapeutic treatment 104.

In one example, the determined motion from surfaces of the patient 102 may be used to confirm body position and accurately track body human motion over time during radiation therapy for radiation oncology applications. Accurately tracking of the surface of the patient 102 is desired for radiation oncology applications as the radiation should be applied carefully to minimize exposure of and collateral damage to healthy tissue. The accurate tracking of respiratory motion is particularly important during radiation therapy as tumors in the lower chest and upper abdomen move as the patient breathes.

Real-time radar imaging of the surface of the patient's skin may be used to monitor motion of the patient 102 during treatment and indicate the most likely position of the target location 106 of the patient 102. High resolution 3D volumetric imaging techniques described herein may be used to provide real time information about not only the respiratory cycle of the patient 102 but also their body's absolute position in space that will allow for real time updates of the position of the patient 102 increasing the effectiveness of the radiation therapy and delivery of the therapeutic treatment 104 to the desired target location 106.

Millimeter-wave (MMW) imaging described herein according to some embodiments of the disclosure is well-suited for tracking body surface as it “sees through” optically opaque clothing. Accordingly, some patients 102 may remain fully-clothed and blanketed while receiving treatment 104 and may reduce the degree of external restraint needed to ensure correct dose delivery.

An antenna system 108 that is incorporated into the medical treatment system 100 is shown in FIG. 8. The antenna system 108 comprises a plurality of transmitters and receivers and different pairs of the transmitters and receivers may be selected during scanning operations as described above with respect to FIG. 2. Antenna system 108 emits electromagnetic energy towards patient 102 and receives electromagnetic energy reflected from patient 102. As shown in the illustrated example embodiment, a beam of therapeutic treatment 104 passes through the antenna system 108 before reaching the patient 102.

The medical treatment system 100 may include additional components such as those shown in FIG. 1 to implement scanning operations of the patient 102 as well as processing of radar data to generate image volumes at different moments in time and processing of the image volumes to determine points of a surface corresponding to the skin of the patient 102. The electromagnetic energy reflected from patient 102 and received by the antenna system 108 may be processed to generate three-dimensional complex-valued image volumes of the patient 102 at different moments in time in accordance with the above-described aspects of the disclosure.

The processing circuitry is further configured to process amplitude information and phase information of the complex values of each of the three-dimensional complex-valued image volumes to generate a plurality of representations, such as point clouds, of the skin of the patient 102 for use to identify a plurality of locations of the target 106 of the patient 102 at the different moments in time. The processing circuitry is configured to use the locations of the target 106 of the patient 102 to control a therapeutic delivery system 110 to direct the therapeutic treatment 104 to the target 106 of the patient 102 at different moments in time of the treatment.

The generated radar images are processed to identify the surface corresponding the skin of the patient 102 at different moments in time when the radar images were generated and the identified surfaces may be used to provide information regarding movement of target location 106 of patient 102 during treatment, for example as discussed above, by registration of point clouds including the target location 106.

Based on radar image derived point cloud data, a patient's breathing cycle can be monitored and the treatment 104 is turned on and off to optimally match the patient's breathing cycle to reduce exposure of healthy tissue to the treatment. In addition, the system 110 can be moved to optimally align with the target location 106 of the patient as their position in space is updated based on the radar image point cloud.

The determined information regarding movement of the patient 102 may be utilized by the medical treatment system 100 to adjust or update the location of where the therapeutic treatment 104 is directed to account for movement of the patient and to attempt to direct the treatment 104 to the target location 106 after movement of the patient 102. The example system 100 of FIG. 8 includes a platform 112 that supports the patient 102 during treatment, a first positioning system 114 and a second positioning system 116. The first positioning system 114 includes one or more motors (not shown) that are configured to move therapeutic delivery system 110 such that a beam of the therapeutic treatment 104 is directed to the target location 106. Accordingly, control of the positioning system 114 enables the direction of the therapeutic treatment 104 to be adjusted during treatment of the patient 102. In addition, the second positioning system 116 includes one or more motors (not shown) that are configured to move platform 112 and patient 102 thereon, and control of the positioning system 116 enables the position of the platform 112 and patient 102 to be adjusted during treatment of the patient 102.

The determined movement of the patient 102 using the radar images discussed above may be used by a microprocessor or other control circuitry to control one or more motors of the positioning systems 114, 116 to direct the therapeutic treatment 104 to the target location 106 of patient 102 as the patient 102 and target location 106 thereof move during treatment and to minimize exposure of other locations of the patient to the therapeutic treatment 104.

As described above, some embodiments of the disclosure utilize phase information in addition to complex amplitude information of a three-dimensional complex-valued image to generate a representation, such as a point cloud, of a surface of a target. The utilization of phase information has increased accuracy with respect to determining the positioning of the surface of the target in space and movement of the surface of the target compared with arrangements that register voxels of different images solely based upon amplitude or intensity that do not necessarily register geometric features of the target between images. Some conventional methods generate surfaces of constant image amplitude without use of phase information which creates substantial errors since the amplitude of these images can vary greatly depending on many factors independent of the target's surface position.

Aspects of the disclosure provide improvements in medical treatment applications, such as radiation oncology applications, since radar images of the patient may be generated through clothing of the patient while some existing systems use optical cameras that cannot adequately handle obscurations such as patient clothing, blankets, or constrainment masks, or these systems use fiducial markers on the skin of the patient. As oncology patients are frequently anemic and hypersensitive to cold temperatures, even a partial disrobing can be very uncomfortable. In addition, some conventional systems use respiratory gating that generally just turns the beam off and on as the lesion or other target moves out of, and back into, the treatment field without redirection of the beam during even a portion of the respiratory cycle of the patient. Some of the systems and method disclosed herein allow a patient to remain fully-clothed and blanketed while receiving radiation therapy and which may also reduce the degree of external restraint needed to ensure correct dose delivery.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended aspects appropriately interpreted in accordance with the doctrine of equivalents.

Further, aspects herein have been presented for guidance in construction and/or operation of illustrative embodiments of the disclosure. Applicant(s) hereof consider these described illustrative embodiments to also include, disclose and describe further inventive aspects in addition to those explicitly disclosed. For example, the additional inventive aspects may include less, more and/or alternative features than those described in the illustrative embodiments. In more specific examples, Applicants consider the disclosure to include, disclose and describe methods which include less, more and/or alternative steps than those methods explicitly disclosed as well as apparatus which includes less, more and/or alternative structure than the explicitly disclosed structure.

1: A surface determination system comprising:
processing circuitry configured to:
access a three-dimensional complex-valued image volume of a target, wherein the three-dimensional complex-valued image volume comprises a plurality of voxels;
define a plurality of image locations of the three-dimensional complex-valued image volume using a first dimension and a second dimension of the three-dimensional complex-valued image volume;
for each of the first locations, identify a plurality of voxels along a third dimension of the three-dimensional complex-valued image volume that correspond to the respective one of the first locations;
for each of the first locations, select one of the voxels that corresponds to the respective one of the first locations as having an increased complex amplitude compared with others of the voxels that correspond to the respective one of the first locations; and
for each of the selected voxels, use phase information of the three-dimensional complex-valued image volume to identify a second location in the third dimension for the respective one of the selected voxels that corresponds to a location of a surface of the target and that is different than a third location of the respective one of the selected voxels.

2: The system of claim 1 wherein the processing circuitry is configured to use the second locations of the selected voxels to generate a representation of the surface of the target.

3: The system of claim 2 wherein the representation of the surface of the target is a point cloud.

4: The system of claim 2 wherein the representation of the surface of the target is a first representation, and the processing circuitry is configured to:
use another three-dimensional complex-valued image volume to generate a second representation of the surface of the target; and
register the first and second representations of the surface of the target with respect to one another to determine movement of the surface of the target.

5: The system of claim 1 wherein the processing circuitry is configured to compare the complex amplitude of each of the identified voxels with respect to a threshold, and to determine that some of the identified voxels do not correspond to the surface of the target as a result of the comparison.

6: The system of claim 5 wherein the processing circuitry is configured to only use the identified voxels having complex amplitudes greater than the threshold to generate the image.

7: The system of claim 1 wherein the second locations of the identified voxels correspond to the surface of the target with increased resolution in the third dimension compared with a resolution of the voxels in the third dimension.

8: The system of claim 1 wherein the three-dimensional complex-valued image volume is a rectangular cuboid, the first and second dimensions correspond to a face of the rectangular cuboid, and the third dimension is in a depth direction of the rectangular cuboid that is perpendicular to the face of the rectangular cuboid.

9: The system of claim 1 wherein the voxels have different phases, and the processing circuitry is configured identify the second location as a result of the phase information of the second location being a given phase value.

10: The system of claim 9 wherein the given phase value is zero-phase.

11: The system of claim 9 wherein the processing circuitry is configured to identify each of the locations as a result of the respective location being closest to one of the selected voxels.

12: The system of claim 9 wherein the complex amplitude data has the given phase value at a plurality of fourth locations in the third dimension for each of the first locations, and the processing circuitry is configured to, for one of the first locations, use a location of the selected voxel having the increased complex amplitude to select one of the fourth locations as the second location that corresponds to the location of the surface of the target.

13: The system of claim 12 wherein the processing circuitry is configured to, for the one of the first locations, select the one of the fourth locations as a result of the selected one of the fourth locations being closest to the location of the voxel having the increased complex amplitude for the one of the first locations.

14: The system of claim 1 wherein each of the identified second locations is between a plurality of the voxels in the third dimension for the respective one of the first locations.

15: The system of claim 1 wherein the processing circuitry is configured to select the voxels having the increased complex amplitude as a result of the selected voxels having maximum complex amplitudes corresponding to each of the first locations.

16: The system of claim 1 wherein the processing circuitry is configured to control emission of electromagnetic energy within a frequency range of 1-100 GHz towards the target, and to process electromagnetic energy reflected from the surface of the target to generate the image volume.

17: The system of claim 1 wherein the second locations more accurately correspond to actual locations of the surface of the target compared with the locations of the voxels having the increased complex amplitude.

18: The system of claim 1 wherein the second locations correspond to respective ones of the first locations in the third dimension.

19: A surface determination system comprising:
an antenna system configured to emit electromagnetic energy towards a target and to receive electromagnetic energy reflected from the target;
processing circuitry configured to:
use the received electromagnetic energy to generate a three-dimensional complex-valued image volume of the target, wherein the three-dimensional complex-valued image volume comprises a plurality of complex values associated with a plurality of voxels; and
use amplitude information and phase information of the complex values of the three-dimensional complex-valued image volume to generate a representation of a surface of the target.

20: The system of claim 19 wherein the representation of the surface of the target is a point cloud.

21: The system of claim 19 wherein the image volume comprises a plurality of voxels, and the processing circuitry is configured to:
use the amplitude information to select a plurality of voxels of the image volume;
use the selected voxels to identify a plurality of locations in the image volume as a result of the phase information for the selected voxels being a given phase value; and
use the identified locations to generate the representation of the surface of the target.

22: The system of claim 21 wherein the given phase value is zero-phase.

23: The system of claim 21 wherein the processing circuitry is configured to select the voxels as a result of the voxels having an increased complex amplitude compared with others of the voxels, and to identify each of the locations as a result of the respective location being closest to one of the selected voxels having the increased complex amplitude.

24: A threat detection system comprising:
an antenna system configured to emit electromagnetic energy towards a clothed individual and to receive electromagnetic energy reflected from the clothed individual;
processing circuitry configured to use the received electromagnetic energy to generate a three-dimensional complex-valued image volume of at least part of the clothed individual, wherein the three-dimensional complex-valued image volume comprises a plurality of complex values associated with a plurality of voxels; and
wherein the processing circuitry is further configured to process amplitude information and phase information of the complex values to generate information regarding a surface anomaly beneath clothing of the clothed individual.

25: The system of claim 24 wherein the processing circuitry is configured to process the amplitude information and the phase information of the complex values to generate a representation of a surface corresponding to the clothed individual's skin.

26: The system of claim 25 wherein the representation is a point cloud.

27: The system of claim 25 wherein the processing circuitry is configured to process the amplitude information of the complex values to identify a plurality of the voxels having an increased complex amplitude compared with others of the voxels, and to use the identified voxels and the phase information of the complex values to identify a plurality of locations within the image volume as corresponding to the representation of the surface.

28: The system of claim 27 wherein the processing circuitry is configured to identify the locations as a result of the locations having a given phase value.

29: The system of claim 28 wherein the given phase is zero-phase.

30: The system of claim 28 wherein the processing circuitry is configured to identify each of the locations as a result of the respective location being closest to one of the voxels having the increased complex amplitude.

31: The system of claim 24 wherein the processing circuitry is configured to generate an image of the surface anomaly.

32: A medical treatment system comprising:
an antenna system configured to emit electromagnetic energy towards a patient and to receive electromagnetic energy reflected from the patient;
processing circuitry configured to use the received electromagnetic energy to generate a plurality of three-dimensional complex-valued image volumes of at least a part of the patient at a plurality of respective moments in time, wherein the three-dimensional complex-valued image volumes individually comprise a plurality of complex values associated with a plurality of voxels; and
wherein the processing circuitry is configured to process amplitude information and phase information of the complex values of each of the three-dimensional complex-valued image volumes to identify a plurality of locations of a target of the patient at the different moments in time; and
wherein the processing circuitry is configured to use the locations of the target of the patient to control a therapeutic delivery system to direct a therapeutic treatment to the locations of the target of the patient at the different moments in time.

33: The system of claim 32 the processing circuitry is configured to process amplitude information and phase information of the complex values of each of the three-dimensional complex-valued image volumes to generate a plurality of representations of skin of the patient, and to use the representations to determine movement of the target of the patient between the different moments in time.

34: The system of claim 33 wherein the representations are point clouds.

35: The system of claim 32 wherein the processing circuitry is configured to process the amplitude information of the complex values to identify a plurality of the voxels each having an increased complex amplitude compared with others of the voxels, and to use the identified voxels and the phase information of the complex values to identify the locations of the target of the patient.

36: The system of claim 35 wherein the processing circuitry is configured to identify the locations of the target of the patient as a result of the phase information of the locations being a given phase value.

37: The system of claim 36 wherein the given phase value is zero-phase.

38: The system of claim 36 wherein the processing circuitry is configured to identify each of the locations of the target of the patient as a result of the respective location being closest to one of the voxels having the increased complex amplitude.

39: The system of claim 32 wherein the target location is on the skin of the patient.

40: The system of claim 32 wherein the therapeutic treatment passes through the antenna system before reaching the patient.
